(19) United States
(12) Patent Application Publication
(10) Pub. No.: US 2016/0120677 A1
(43) Pub. Date: May 5, 2016

(54) METHOD AND SYSTEM FOR CONTROLLED STENT DEPLOYMENT and RECONSTRAINT

(71) Applicant: Flexible Stenting Solutions, Inc., Eatontown, NJ (US)

(72) Inventors: Taylor A. Heanue, Oakland, CA (US); Diana Lui, Berkeley, CA (US); William David Kelly, Los Altos, CA (US)

(73) Assignee: Flexible Stenting Solutions, Inc., Eatontown, NJ (US)

(21) Appl. No.: 14/931,295

(22) Filed: Nov. 3, 2015

Related U.S. Application Data
(60) Provisional application No. 62/074,609, filed on Nov. 3, 2014.

(51) Int. Cl. A61F 2/966; A61F 2/844

(52) U.S. Cl. CPC A61F 2/966; A61F 2/844; A61F 2002/9534

(57) ABSTRACT

A medical device delivery system including a mechanism to concurrently move an inner member and an outer member in opposite directions and at pre-set speed ratio can be operated, for example, to reconstrain a foreshortening self-expanding stent with a known foreshortening ratio between the crimped diameter in an intraluminal catheter based delivery system and the nominal deployed diameter in the body lumen. The mechanism can include two oppositely handed lead screws that concurrently turn and two followers, each follower operatively connected to one of the two shafts (e.g., the inner and outer member).

### RELATED APPLICATIONS

This application claims the benefit of priority to U.S. Provisional Patent Application No. 62/074,609, filed Nov. 3, 2014, which is expressly incorporated in its entirety.

### BACKGROUND

1. Technical Field

The invention relates to the field of medical devices, and more particularly medical devices or delivery systems for and methods of controllably deploying stents and reconstraining partially deployed stents.

In some applications, the invention relates to systems for delivering a self-expandable intraluminal graft (“stents”) for use within a body passageway or duct which are particularly useful for repairing blood vessels narrowed or occluded by disease.

2. Related Devices and Methods

Transluminal prostheses have been widely used in the medical arts for implantation in blood vessels, biliary ducts, or other similar lumens of the living body. These prostheses are commonly known as stents and are used to maintain, open, or dilate tubular structures. An example of a commonly used stent is given in U.S. Pat. No. 4,733,665 filed by Palmaz on Nov. 7, 1985, which is hereby incorporated in its entirety herein by reference. Such stents are often referred to as balloon expandable stents. Typically the stent is made from a solid tube of stainless steel. Thereafter, a series of cuts are made in the wall of the stent. The stent has a first smaller diameter which permits the stent to be delivered through the human vasculature by being crimped onto a balloon catheter. The stent also has a second, expanded diameter, upon the application, by the balloon catheter, from the interior of the tubular shaped member of a radially, outwardly extending force.

However, such stents are often impractical for use in some vessels such as the carotid artery or the superficial femoral artery. The carotid artery is easily accessible from the exterior of the human body, and is often visible by looking at one's neck. A patient having a balloon expandable stent made from stainless steel, or the like, placed in his or her carotid artery might be susceptible to severe injury through day-to-day activity. A sufficient force placed on the patient's neck, such as by falling, could cause the stent to collapse resulting in injury to the patient. In order to prevent this and to address other shortcomings of balloon expandable stents, self-expanding stents were developed. Self-expanding stents act like springs and will recover to their expanded or implanted configuration after being crushed.

One type of self-expanding stent is disclosed in U.S. Pat. No. 4,665,771, which stent has a radially and axially flexible, elastic tubular body with a predetermined diameter that is variable under axial movement of ends of the body relative to each other and which is composed of a plurality of individually rigid but flexible and elastic thread elements defining a radially self-expanding helix. This type of stent is known in the art as a “braided stent” and is so designated herein.

Other types of self-expanding stents use alloys such as Nitinol (Ni—Ti alloy) which have shape memory and/or superelastic characteristics in medical devices that are designed to be inserted into a patient's body. The shape memory characteristics allow the devices to be deformed to facilitate their insertion into a body lumen or cavity and then be heated within the body so that the device returns to its “memorized” shape. Superelastic characteristics on the other hand generally allow the metal to be deformed and restrained in the deformed condition to facilitate the insertion of the medical device containing the metal into a patient's body, with such deformation causing the phase transformation. Once within the body lumen the restraint on the superelastic member can be removed, thereby reducing the stress therein so that the superelastic member can return to its original un-deformed shape by the transformation back to the original phase, or close to it (as the implanted shape is designed to have some deformation to provide a force to prop open the vessel in which it is implanted).

Alloys having shape memory/superelastic characteristics generally have at least two phases. These phases are a martensitic phase, which has a relatively low tensile strength and which is stable at relatively low temperatures, and an austenitic phase, which has a relatively high tensile strength and which is stable at temperatures higher than the martensitic phase.

When stress is applied to a specimen of a metal such as Nitinol exhibiting superelastic characteristics at a temperature above which the austenite is stable (i.e. the temperature at which the transformation of martensitic phase to the austenite phase is complete), the specimen deforms elastically until it reaches a particular stress level where the alloy then undergoes a stress-induced phase transformation from the austenitic phase to the martensite phase. As the phase transformation proceeds, the alloy undergoes significant increases in strain but with little or no corresponding increases in stress. The strain increases while the stress remains essentially constant until the transformation of the austenite phase to the martensite phase is complete. Thereafter, further increase in stress is necessary to cause further deformation. The martensitic metal first deforms elastically upon the application of additional stress and then plastically with permanent residual deformation.

If the load on the specimen is removed before any permanent deformation has occurred, the martensitic specimen will elastically recover and transform back to the austenite phase. The reduction in stress first causes a decrease in strain. As stress reduction reaches the level at which the martensitic phase transforms back into the austenite phase, the stress level in the specimen will remain essentially constant (but substantially less than the constant stress level at which the austenite transforms to the martensite) until the transformation back to the austenite phase is complete, i.e. there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as superelasticity or pseudoelasticity. It is this property of the material which makes it useful in manufacturing tube cut self-expanding stents. The prior art makes reference to the use of metal alloys having superelastic characteristics in medical devices which are intended to be inserted or otherwise used within a patient's body. See for example, U.S. Pat. No. 4,665,905 (Jervis) and U.S. Pat. No. 4,925,445 (Sakamoto et al.).

A now conventional delivery system for a self-expanding stent is a so-called “pin and pull” system. The following is an example of a “pin and pull” system. The delivery system includes an outer sheath, which is an elongated tubular member having a distal end and a proximal end and a lumen therethrough. A typical outer sheath is made from an outer polymeric layer, an inner polymeric layer, and a braided reinforcing layer between the inner and outer layers. The reinforcing layer is more rigid than the inner and outer layers. It is this outer sheath which is “pulled” in the “pin & pull” system. The “pin & pull” system further includes an inner shaft located coaxially within the outer sheath. The shaft has a distal end, extending distal of the distal end of the sheath, and a proximal end, extending proximal of the proximal end of the sheath. It is this shaft which is “pinned” in the “pin & pull” system. A “pin & pull” system further has a structure to limit the proximal motion of the self-expanding stent relative to the shaft. This “stent stopping” structure is located proximal to the distal end of the sheath. Lastly, a “pin & pull” system includes a self-expanding stent located within the sheath. The stent in its reduced diameter state for delivery makes frictional contact with the inner diameter of the outer sheath, more specifically, with the inner diameter of the inner layer of the outer sheath. The stent is located between the stop structure and the distal end of the sheath, with a portion of the shaft disposed coaxially within a lumen of the stent. The stent makes contact with the stop structure during deployment as the sheath is withdrawn and moves the stent with it (due to the frictional contact between the stent and the inner diameter of the sheath). The proximal motion of the proximal end of the stent is stopped as it comes into contact with the stop structure and the stop structure provides a counteracting force on the stent, equal and opposite to the frictional force from the sheath on the stent.

To deploy a stent from a “pin & pull” system, the system is navigated to the treatment location. Then the inner shaft, which extends proximal of the proximal end of the outer sheath is held fixed against the patient with one hand of the operator (medical professional). This action fixes the location of the inner shaft along a longitudinal axis of the patient's lumen being stented. This action is the “pin” step in the “pin & pull” system. The physician takes his or her other hand and pulls the outer sheath proximally (drawing some of it out of the patient toward the “pinning” hand) to unconstrain, expose, and deploy the stent. This action is the “pull” step in the “pin & pull” system.

An early example of another “pin & pull” system is the Gianturco stent delivery system as described in U.S. Pat. No. 4,580,568 issued Apr. 8, 1986. In this prior art delivery system, the outer sheath is a tube of a single material, which does not have a reinforcing structure within it. A cylindrical flat end pusher, having a diameter almost equal to the inside diameter of the sheath is inserted into the sheath behind the stent. The pusher or inner shaft is then used to push the stent from the proximal end of the sheath to the distal end of the sheath. Deployment of the stent is accomplished by holding the inner shaft fixed with respect to the patient's body and pulling back on the sheath to expose the stent, which expands upon removal of the radially restraining force, as illustrated in FIGS. 4 & 5 of U.S. Pat. No. 4,580,568, which are incorporated herein by reference.

Another early self-expanding stent on the market was the Wallstent. It was braided and changed both length, which shortened, and diameter, which increased, when it was deployed, and the change to its length was appreciable. U.S. Pat. No. 4,655,771 to Wallsten, herein after “Wallsten”, describes a couple of delivery systems for a braided stent, called a “tubular body” in the patent. One of the delivery systems is illustrated in FIG. 11 of Wallsten, which is described as follows, “[i]In FIG. 11 there is shown another embodiment of the assembly for use in expanding the tubular body. This assembly constitutes a flexible instrument intended to introduce the tubular body in contracted state into for example a blood vessel and then to expand the body when located therein. The parts of the instrument consist of an outer flexible tube 61 and a concentric also flexible inner tube 62. At one end of the outer tube an operational member 63 is arranged. Another operational member 64 is attached to the free end of inner tube 62. In this manner the inner tube 62 is axially displaceable in relation to the outer tube 61. At the other end of inner tube 62 a piston 65 is attached which when moving runs along the inner wall of outer tube 61. When the instrument is to be used the tubular expansible body 69 in contracted state is first placed inside tube 61, the inner tube 62 with the piston 65 being located in the rear part 66 of outer tube 61. The starting position of piston 65 is shown by dashed lines at 67 in FIG. 11. In this manner part of tube 61 is filled with the contracted tubular body 69 in the starting position. During implantation the flexible tubular part of the device is inserted to the location of a blood vessel intended for implantation. Member 64 is then moved in the direction of arrow 68, the contracted body 69 being pushed out through end 70 of tube 61, the part of the tubular body 69 leaving tube end 70 expanding until in its expanded position 71 it is brought to engagement with the interior of vascular wall 72. The tubular body 69, 71 is for sake of simplicity shown in FIG. 11 as two sinus-shaped lines. To the extent that the expanded body 21 comes into engagement with vascular wall 72 tube end 70 is moved by moving member 63 in the direction of arrow 73. The contracted body 69 is moved by the piston 65 pushing against one end of the body. Thus, the implantation takes place by simultaneous oppositely directed movements of members 64 and 63, the displacement of member 64 being larger than that of member 63.” Like the delivery system for the Gianturco stent, its sheath was not reinforced, but was a single material tube, and its inner shaft did not extend through the stent, but terminated at the proximal end of the stent constrained at the distal end of the outer sheath. The inner shaft was coaxial with the outer sheath, and had an outer diameter that was larger than the inner diameter of the reduced diameter “constrained” or crimped stent.

Many conventional self-expanding stents are designed to limit the stent foreshortening to an amount that is not appreciable (e.g., less than 10%). Stent foreshortening is a measure of change in length of the stent from the crimped or radially compressed state as when the stent is loaded on or in a delivery catheter to the expanded state. Percent foreshortening is typically defined as the change in stent length between the delivery catheter loaded condition (crimped) and the nominal deployed diameter (i.e., the labeled diameter which the stent is intended to have when deployed, i.e., a “10 mm stent” has a nominal deployed diameter of 10 mm.) divided by the length of the stent in the delivery catheter loaded condition (crimped), multiplied by 100. Stents that foreshorten an appreciable amount (e.g., equal to or more than [insert a value here]) can be more difficult to deploy where intended axially when being deployed in a body lumen or cavity, such as a vessel, artery, vein, or duct. The distal end of the stent has a tendency to move in a proximal direction as the stent is being deployed in the body lumen or cavity. And, in conditions where the distal end is stationary with respect to the vessel wall, the proximal end of the stent will move distally as a function of the foreshortening upon expansion. Thus foreshortening may lead to a stent being placed in an incorrect or suboptimal location. Delivery systems that can compensate for stent foreshortening would have many advantages over delivery systems that do not.

When a self-expanding stent is deployed in the vessel in an unintended location, an additional stent may be required to cover the full length of the diseased portion of the vessel, and some stent overlap may occur. Obviously, the ability to reposition a stent to correctly deploy it in the intended location is preferred. Often, repositioning a stent requires that the stent first be reconstrained within the outer tubular member of the delivery system (often referred to as a “sheath”). To reconstrain a stent, the outer tubular member is pushed distally to slide over the stent and radially compress it back to its crimped diameter. To resist the axial force of the sheath on the stent due to friction, the proximal end of the stent which is still in the sheath is typically restrained from distal motion relative to the sheath and inner member. A number of delivery system designs provide features to restrain the proximal end of the stent from distal motion, see, e.g., U.S. patent application Ser. No. 12/573,527, Attorney docket number FSS5004USNP, filed Oct. 5, 2009, and Ser. No. 13/494,567, Attorney docket number FSS5004USCIP, filed Jun. 12, 2012, and European Patent Publication No. 0696442 A2, and U.S. Patent Publication No. 2007/0233224 A1.

### SUMMARY OF THE INVENTION

One aspect of the invention is a method of reconstraining a partially deployed self-expanding stent that uses a mechanism to move the inner shaft and the outer tubular member in opposite directions at rates that are proportional to each other in accordance to the foreshortening ratio of the stent being reconstrained.

Another aspect of the invention is a number of hand or motor actuated mechanisms that may be actuated to perform the above method.

One invention described and claimed herein is a method of reconstraining a foreshortening self-expanding stent with a known foreshortening ratio between the crimped diameter in an intraluminal catheter based delivery system and the nominal deployed diameter in the body lumen, wherein the proximal end of the stent is in releasable fixed relation about a location along the length of an inner member of a stent delivery system, the method comprising translating proximally the outer member with respect to the stent at a first rate, thereby exposing at least a portion of the stent, at the same time that the outer member is translating proximally, translating distally the inner member, thereby translating distally the proximal end of the stent at a rate equal to the known foreshortening ratio multiplied by the first rate at which the outer member is translating proximally, after exposing at least a length of the stent, but before translating proximally the distal end of the outer member past the proximal end of the stent, deciding to reconstrain the partially deployed stent, subsequently translating distally the outer member with respect to the stent at a second rate, thereby reconstraining the length of the stent exposed in the previous translating proximally step, and at the same time that the outer member is translating distally, translating proximally the inner member at a rate equal to the known foreshortening ratio multiplied by the second rate at which the outer member is translating distally.

Another invention described and claimed herein is a medical device delivery system comprising a first lead screw having a right-handed thread and a central longitudinal axis, a second lead screw having a left-handed thread and a central longitudinal axis, a first follower operationally coupled to the right-handed thread to translate without rotating, a second follower operationally coupled to the left-handed thread to translate without rotating, wherein when the first and second lead screws rotate, the first follower translates parallel to the central longitudinal axis of the first lead screw in a first linear direction and the second follower translates parallel to the central longitudinal axis of the second lead screw in a linear direction opposite the first linear direction.

Yet another invention described and claimed herein is a medical device delivery system comprising a first lead screw having a right-handed thread and a central longitudinal axis, a second lead screw having a left-handed thread and a central longitudinal axis, a first follower operationally coupled to the right-handed thread to translate without rotating, a second follower operationally coupled to the left-handed thread to translate without rotating, wherein the central longitudinal axis of the first and second lead screws are on a common line and are coupled together to rotate about the common line in the same rotational direction and at the same time, such that when the first and second lead screws rotate, the first follower translates parallel to the common line in a first linear direction and the second follower translates parallel to the common line in a linear direction opposite the first linear direction.

These and other features, benefits, and advantages of the present invention will be made apparent with reference to the following detailed description, appended claims, and accompanying figures, wherein like reference numerals refer to structures that are either the same structures, or perform the same functions as other structures, across the several views.

### BRIEF DESCRIPTION OF THE FIGURES

The figures are merely exemplary and are not meant to limit the present invention.

FIG. 1 illustrates a stent delivery system;

FIG. 2A illustrates a self-expanding stent in a constrained diameter and length;

FIG. 2B illustrates a self-expanding stent in a nominal deployment diameter and length;

FIG. 3 illustrates an assembly of two lead screws and two followers;

FIG. 4 illustrates the assembly of FIG. 3 connected to two elongated members;

FIG. 5 illustrates a side view of a handle of a medical device delivery system including an embodiment of one aspect of the present invention;

FIG. 6 illustrates a front view of the handle of FIG. 5;

FIG. 7 illustrates a front view of another embodiment of one aspect of the present invention;

FIG. 8 illustrates a front view of yet another embodiment of one aspect of the present invention;

FIG. 9 illustrates a partial side view of the embodiment of FIG. 8;

FIG. 10 illustrates a front view of fourth embodiment of one aspect of the present invention;

FIG. 11 illustrates a partial side view of the embodiment of FIG. 10;

FIG. 12 illustrates a front view of an embodiment of a follower;

FIG. 13 illustrates a front view of an embodiment of a follower with bearings;

FIG. 14 illustrates a side view of yet another alternative embodiment of the mechanism, in which the first and second lead screws can have their central longitudinal axes parallel to one another; and

FIG. 15 illustrates a front view of the embodiment of FIG. 14.

### DETAILED DESCRIPTION

As used herein, “foreshortening ratio” is defined as the result of dividing the value of the length of the nominal diameter stent subtracted from the length of the crimped diameter stent by the length of the crimped diameter stent.

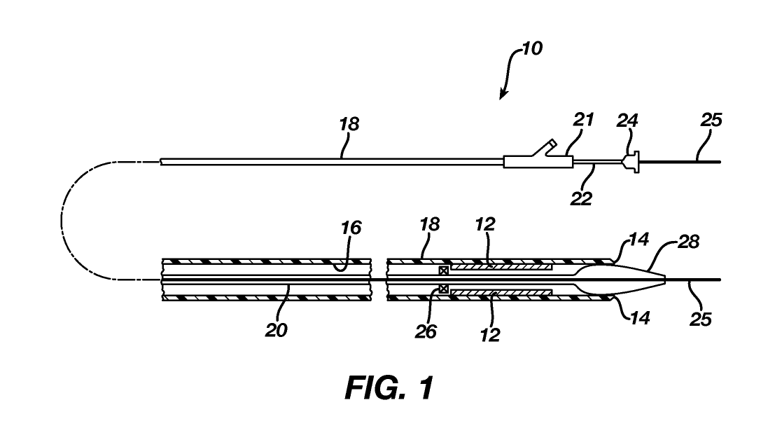

In FIG. 1, a stent delivery system 10 includes a self-expanding stent 12 at the distal end 14 of the lumen 16 of a flexible tubular member 18, which surrounds a smaller diameter flexible tubular member 20. Each of the tubular members is connected to a hard plastic structure (21, 24), which serves, among other functions, as the piece with which to manipulate the tubular member. At the proximal end, the smaller diameter flexible tubular member 20 is connected to a stiffer tubular member 22, which may be a hypotube, and the grip or handle 24 is connected to the proximal end of the hypotube 22. Stiffer tubular member 22 and flexible tubular member 20 may have a lumen for tracking over a guidewire 25. Structure 26 mounted on flexible tubular member 20 functions to keep stent 12 is releasable fixed relation to a longitudinal point on the length of tubular member 20. Finally, stent delivery system may include a distal tip that is distal to the distal end of flexible tubular member 18 and acts as a dilator when entering the body, a blood vessel in particular.

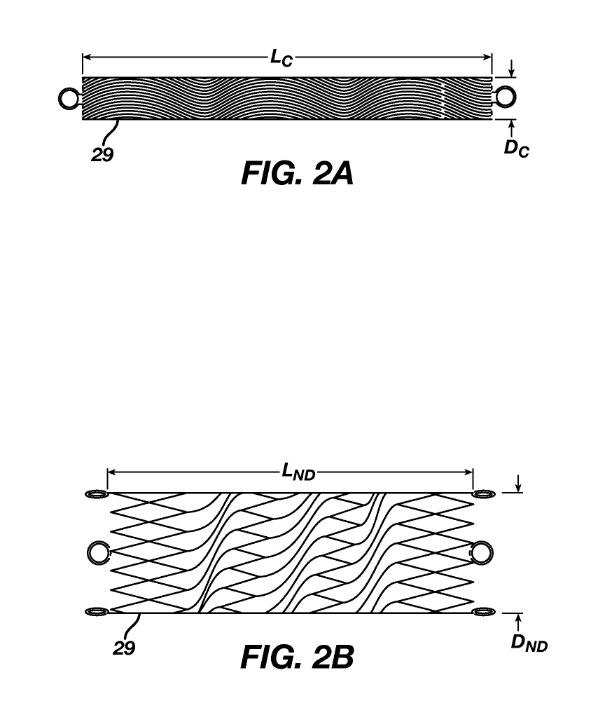

The stents that are delivered to the treatment location may be self-expanding. FIG. 2A is a schematic representation of a fully connected, helical geometry self-expanding stent 29 in a state of crimped diameter and length. This is the state of the stent when completely constrained in the lumen of the outer tubular member of the stent delivery system. FIG. 2B is a schematic representation of the same stent 29 in the nominal deployed state, which has a larger diameter and a shorter length. The difference between the crimped length and the nominal deployed length is considered significant if it is greater than 10%. When deployed, if the distal end of the stent contacts the vessel wall when it expands, the distal end is then stationary with respect to the vessel. In these conditions, the proximal end of the stent must move distally from that time on to permit the stent to expand as it deploys.

Reconstraining includes pushing the outer tubular member distally to slide over the expanded stent until the tubular member constrains the entire length of the stent and the stent is no longer in contact with the vessel wall, and can be repositioned without risk of stretching the vessel which may lead to injury. Just as the proximal stent stop applied counteracting distal forces to the proximal end of the stent to counteract the proximal friction forces along the outer diameter of the stent in contact with the proximally translating outer tubular member, and allowed the tubular member to be withdrawn to expose the stent, a structure is needed to apply proximally acting forces to the stent to counteract the distally acting friction forces of the distally translating tubular member on the outer diameter of the stent. If insufficient counteracting force is provided, when the tubular member is advanced distally, since the distal end of the stent is in contact with the vessel wall, which resists distal motion, one possible outcome is that the tubular member does not slide over the stent, such that the portion of the stent that is exposed and unconstrained begins to evert around the advancing distal end of the tubular member as the constrained portion of the stent at a smaller diameter is advanced toward a relatively stationary expanded diameter distal end of the stent. Systems are known in the art for providing structures to provide such a counteracting proximal force, and examples are U.S. patent application Ser. No. 12/573,527, Attorney docket number FSS5004USNP, filed Oct. 5, 2009, (a rotatable band which interfaces with the inner diameter of the crimped stent, protruding through it and holding that part of the stent in place, when against a stop on the inner shaft) and Ser. No. 13/494,567, Attorney docket number FSS5004USCIP, filed Jun. 12, 2012, (a rotatable stent lock with has axially extending protrusions that interface with the proximal end of the stent at the same radial location as the crimped stent, when against a stop on the inner shaft) and European Patent Publication No. 0696442 A2 (four radially projecting members fixes to the inner shaft which mechanically interfere with axial motion of the crimped stent (proximal or distal)), and U.S. Patent Publication No. 2007/0233224 A1 (rotatable, but axially fixed (to the inner shaft) bumpers that stick to the inner diameter of the crimped stent). However, when a stent has an appreciable (relative to the length of the section of the vessel being treated) increase in length upon constraining (or, i.e., crimping), proximal motion of the structure that provides these counteracting forces may provide optimal conditions for reconstraining a stent.

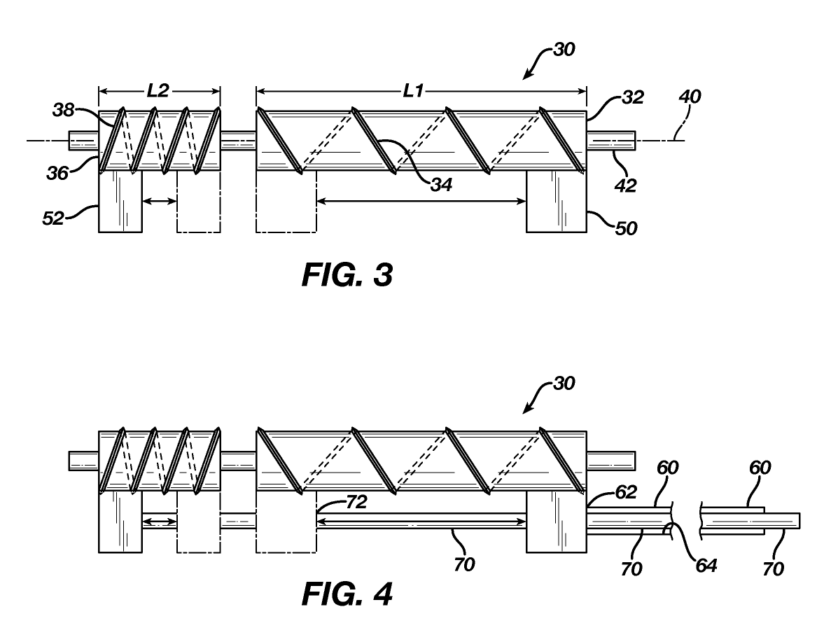

FIG. 3 illustrates a side view of a mechanism 30 that can provide constant ratio relative motion by either advancing the inner tubular member while retracting the outer tubular member (for exposing and deploying a stent) or by alternatively retracting the inner tubular member while advancing the outer tubular member (for reconstraining a partially deployed stent). Thus when the proximal end of the stent is fixed longitudinally with respect to the longitudinal axis of the inner tubular member, the proximal end of the stent is translated the expected distance to account for the expected foreshortening distally upon deployment or forelengthening proximally into the outer tubular member during reconstraining. Turning to mechanism 30, it includes a first lead screw 32 with a helical thread 34 over length L1. In the illustrated mechanism, helical thread 34 is right handed and has a predetermined pitch. Mechanism 30 includes a second lead screw 36 with a helical thread 38 over length L2. In the illustrated mechanism, helical thread 38 is left handed and has a predetermined pitch. First and second lead screws both have central longitudinal axes which are axially aligned along a common line 40. In the illustrated mechanism 30, first and second lead screws are fixedly connected to a smaller diameter shaft 42, used for mounting the assembly of lead screws to a frame (not shown). Mechanism 30 includes a first follower 50, illustrated in FIG. 3 as a square. First follower 50 interfaces with lead screw 32 and when constrained from rotating, translates parallel to common line 40, when lead screw 32 rotates. Mechanism 30 includes a second follower 52, illustrated in FIG. 3 as a square. Second follower 52 interfaces with lead screw 36 and when constrained from rotating, translates parallel to common line 40, when lead screw 36 rotates. Initial positions of followers 50 and 52 are depicted in solid lines and final positions are depicted in broken lines. Arrows illustrate the translation parallel to common line 40 between the initial and final positions. The ratio of the pitches of the helical threads is, in the depicted embodiment, equal to the ratio of L1 to L2. In FIG. 3, it can be seen that followers 50 and 52 move in opposite directions, and at different rates given the same rotational input of their respective lead screw.

Mechanism 30 can be operated to translate at the same time two members in opposite directions at different rates with a single rotational input. In FIG. 4, mechanism 30 is illustrated connected to two elongated tubular members. The first elongated tubular member 60 is operatively connected to follower 50 at its distal end 62. As illustrated elongated tubular member 60 is hollow and has a lumen 64. A second elongated tubular member 70 is operatively connected with follower 52 at its proximal end 72. Elongated tubular member 70 has a smaller outer diameter than the inner diameter of elongated tubular member 60, and as illustrated, a length less than the total length of 70 is inside the lumen 64 and co-axial with elongated tubular member 60. When shaft 42 is rotated, follower 50 will translate proximally and elongated member 60 will translate an equal amount at the same time due to the operative connection between them. When shaft 42 is rotated, follower 52 will translate distally and elongated member 70 will translate an equal amount at the same time due to the operative connection between them.

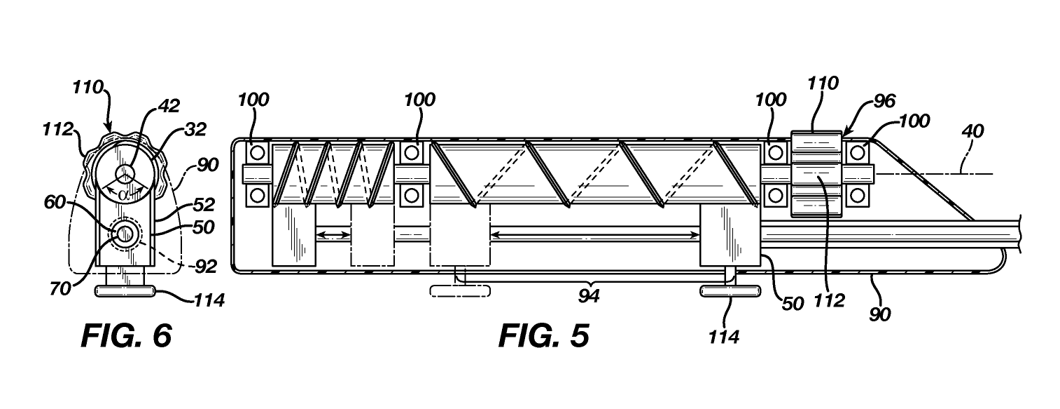

FIG. 5 illustrates the assembly of mechanism 30 and elongated members 60 and 70 in half of a housing 90. Housing 90 substantially encloses mechanism 30, in addition to enclosing the proximal portions of elongated members 60 and 70. Housing 90 defines opening 92 at its distal tip for the elongated members 60 and 70 to translate through. Housing 90 defines an opening 94 for a portion of a follower that may be used as an input 114 to the system by manipulation by a user. In some embodiments, opening 94 is a straight slot. Housing 90 defines an opening 96 to accommodate a rotatable input 110 operatively connected to shaft 42. Shaft 42 is mounted in bearings 100 to housing 90. In some embodiments, not depicted, housing 90 defines additional openings. In some embodiments of the present invention, housing 90 functions as a handle to a medical device delivery system. In some embodiments of the present invention, housing 90 is sized to be grasped by a human hand. Such sizing does not necessarily impact the length of housing 90, just the circumference of a transverse cross section to common line 40 (like shown in FIG. 6). As housing 90 substantially encloses mechanism 30, mechanism 30 is accordingly sized to housing 90.

Input 110 as illustrated in FIG. 5 is a short cylinder with a knurled or otherwise grippable surface, for example, using facets 112 about the generally cylindrical circumference. It is envisioned that an operator of mechanism 30 may use a thumb or finger to apply tangential force to input 110 to rotate it about common line 40. Input 110 is operatively connected to the two lead screws, such that rotation of input 110 results in rotation (in the same direction) of lead screws 32 and 36, and translation of followers 50 and 52, and translation of elongated members 60 and 70. The larger the diameter of input 110, the greater the mechanical advantage to operate the mechanism.

In the illustrated embodiment of FIGS. 5 & 6, mechanism 30 is configured such that follower 50 can be used as an input to the system. To accommodate such manipulation of follower 50 in embodiments with a housing, follower 50 is configured to project through opening 94 to present a tab or other suitable structure for a user to manipulate by translation within opening 94. Such structure is alternatively referred to herein as an input 114. If a user translates input 114, lead screw 50 rotates, resulting in lead screw 52 rotating in the same direction as lead screw 50, follower 52 translating in an opposite direction from the input translation, and input 110 rotating in the same direction as lead screw 50. Of course, due to the operative connections of elongated tubular members to the respective followers, translating input 114 will also translate the elongated members in opposite directions.

Gripping the outer elongated tubular member outside of the housing and translating it along its longitudinal axis is, in some embodiments, an acceptable input to the mechanism as well, resulting in the translation of the follower to which it is operatively connected to translate in the same direction, rotating the first lead screw, and producing the rest of the motions the mechanism is configured to produce as described above.

Thus, in some embodiments of a device incorporating such a mechanism 30, a user may achieve the desired exposure of a constrained stent or reconstraint of a partially deployed stent by rotating input 110, translating input 114, or translating outer tubular member 60 external to the housing 90 and patient in the desired direction to accomplish the desired exposure or reconstraint.

FIG. 6 illustrates a front view of the complete housing in phantom lines, and the input 110, shaft 42, follower 50, input 114, follower 52 and elongated tubular members 60 and 70 to show other aspects of mechanism 30. In the illustrated embodiment, a follower interfaces with its respective lead screw over an internal angle alpha, α, of less than 180 degrees, and more closely approximating 90 degrees. As long as the follower interfaces sufficiently with the threads of the lead screw, such an angle measurement over which the two parts are in contact is not necessary. Alternatively, followers 50 and 52 could be annular rings, like a nut, about and co-axial with the lead screw and its longitudinal axis, here the common line 40. The follower must be prevented from rotating, so that elongated tubular members can translate in a straight line through housing 90 and out opening 92. Another aspect illustrated in FIG. 6 is the portion of input 110 which extends through opening 96 in housing 90. Here the knurled or faceted ring-like surface of input 110 may be manipulated by a user's thumb or finger for one handed operation (i.e., hold the handle and rotate input 110 with the thumb of the same hand, or by one or more digits on the hand not holding the handle for two handed operation via input 110. FIG. 6 also illustrates input 114 extending through opening 94 to provide a structure that can be manipulated by the user to translate (in and out of the page in the view of FIG. 6) to actuate mechanism 30 and provide opposite and scaled translation between the two tubular members of the device.

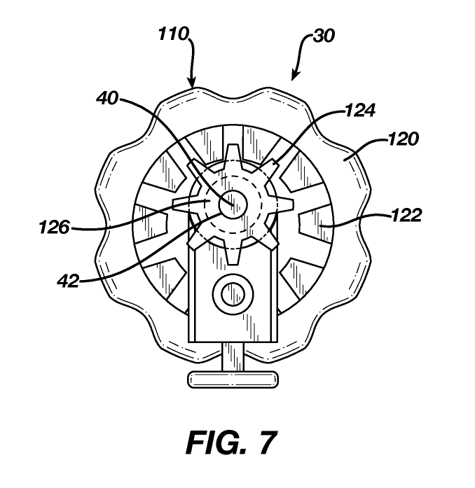

Another embodiment of a rotatable input (with respect to the housing 90) is illustrated in FIG. 7, which is another front view, to most easily show difference between this embodiment and the last. Here input 110 is an internal gear 120 with a larger diameter than the short cylinder illustrated in FIGS. 5 and 6. The internal gear 120 has teeth 122 that engage mating teeth 124 of a spur gear 126 located within the internal opening of the internal gear 120. Spur gear 126 is axially aligned with common line 40 and is operatively connected to lead screw 50 (and the rest of mechanism 30). Thus a greater mechanical advantage is obtained using the illustrated embodiment, and all other things being the same about mechanism 30, fewer rotations of input 110 are needed to fully expose or reconstrain a stent with a delivery system including this embodiment.

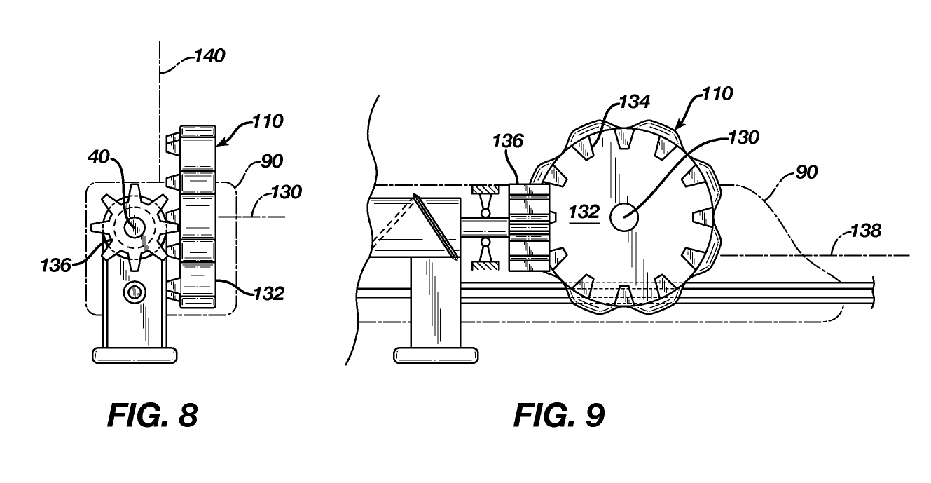

Yet another embodiment of rotatable input 110 is illustrated in a front view in FIG. 8 and a partial side view in FIG. 9. This input to the mechanism rotates about an axis 130 that is perpendicular to the common line 40, and relies on a face gear 132, that is, one with teeth 134 projecting along the axis 130 of the gear off of one “face” of the gear 132, rather than projecting radially inward (as in an internal ring gear) or radially outward (as in an external ring gear). Here again, housing 90 is drawn in phantom lines to more clearly see arrangement of new components. Face gear 132 engages with a spur gear 136, the same as or similar to the one illustrated in FIG. 7, but the user interface is different. Instead of rotating the input 110 across the handle, a user rotates the input 110 in-line with the longitudinal axis of the handle. As illustrated, the rotatable input 110 would be on one lateral side or the other with respect to the longitudinal midplane 140 of the handle.

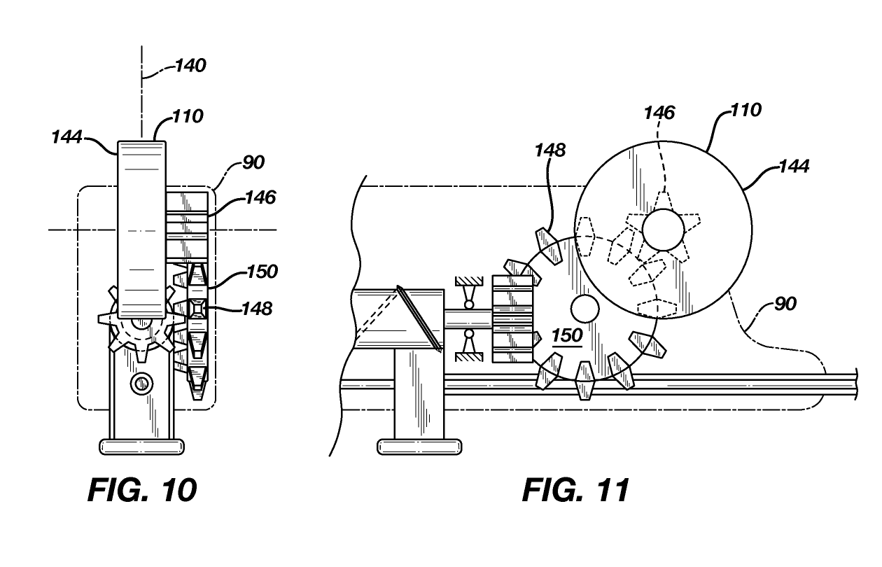

Yet another embodiment of rotatable input 110 is illustrated in a front view in FIG. 10 and a partial side view in FIG. 11 to illustrate differences between this embodiment and the others. This embodiment builds on the last embodiment by incorporating an “in-line” rotatable input 110 on the handle, but additionally, it centers the input 110 along the longitudinal midplane 140 of the handle. This requires an additional rotatable structure, here the combination of a knurled short cylinder 144 fixedly connected to a spur gear 146. The face gear of the last embodiment additionally must have external teeth 148 with which to engage the spur gear 136, thus being a combination face and external gear 150. The housing 90 and gears can be sized to optimize the desired ease of handling and gear ratio between the input and the gears in the chain (here 146, 150, and 136) that operate mechanism 30 and result in opposite movement of the two tubular members operatively connected to the followers.

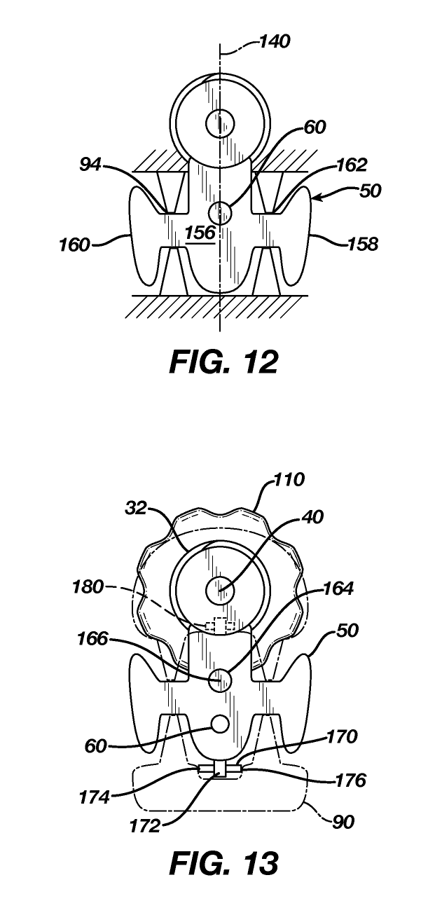

A follower that is also going to function as a translatable input to the mechanism can have different forms than depicted in FIGS. 5-11. FIG. 12 illustrates a front view of a follower 156 that provides a projection (158, 160) laterally on either side of a vertical midplane 140 of the handle. Housing 90 is accordingly adjusted moving opening 94 from the “bottom” of the handle to a side and also defining an additional opening 162 for the lateral projection on the opposite side of the follower. That way, translating the lateral projection of the follower on either side of the handle can be used to actuate mechanism 30 and provide translation in opposite directions of the two elongated tubular members operatively connected to the two followers.

And an additional design option for operation requiring less actuating force is illustrated in FIG. 13, which illustrates the incorporation of bearings into a mechanism utilizing followers similar to that illustrated in FIG. 12. In this embodiment, followers 50 and 52 define an additional through-hole 164 which is a bearing surface against a bearing rod 166, which runs parallel to common line 40. Additionally, a round bearing 170, the inner race of which surrounds a vertical post 172 extending down from the follower 50, counteracts the moment exerted on the follower 50 from the rotation of the lead screw 32. The lower bearing 170 rotates against one of two vertical walls 174, 176 provided in housing 90 to prevent rotation of follower 50.

In order to reduce system friction, it may be desirable to exchange the “threads” of lead screw and follower with more of a cam-follower setup. In this embodiment, follower 50 contains a bearing in contact with it and the leadscrew, which now longer is strictly a lead screw (as there are not interfacing grooves, i.e., mating threads, in follower 50). Instead structure 50 is actually a helical cam for that bearing to follow.

Reducing system friction to negligible amounts increases efficiency and allows backdriving so that translation of translatable input 114 can rotate lead screw 32. The cam/bearing method is one way to achieve this. Also a ball nut could be used or simply very low friction materials, lubricants, etc.

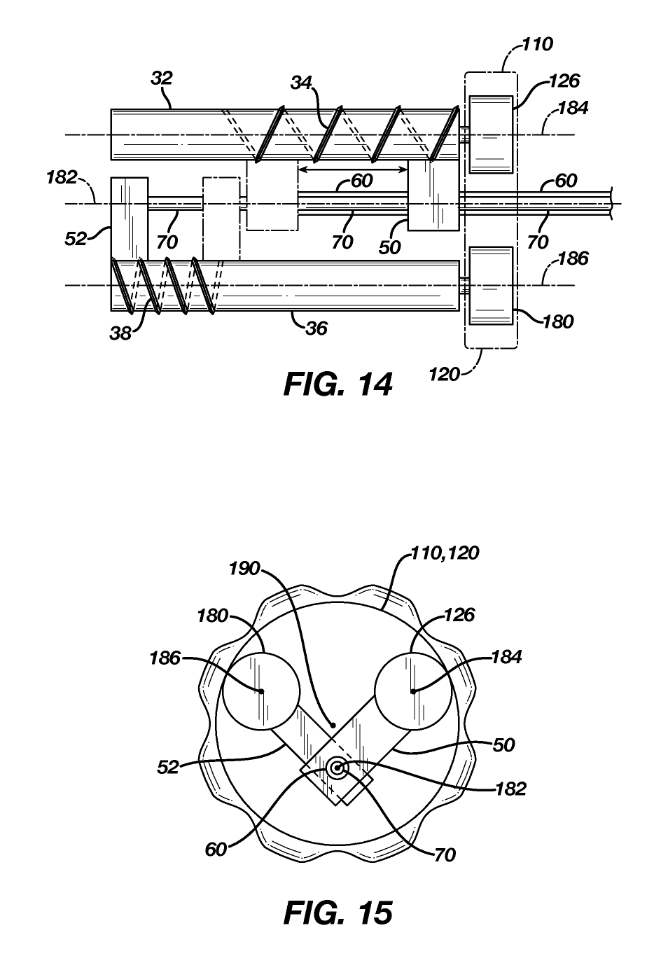

FIGS. 14 and 15 illustrate an alternative embodiment of the mechanism, in which the first lead screw 32 and second lead screw 36 have parallel central longitudinal axes (184, 186), rather than axially aligned ones. The elongated members 60, 70 attached to the first and second followers 50, 52 have a common central longitudinal axis 182 parallel to each of the respective central longitudinal axis of the first and second lead screw. In such an embodiment, a single rotatable input 110 may be an internal ring gear 120 engaging with two spur gears 126, 180, one for each of the two parallel lead screws, similar to the embodiment depicted in FIG. 7. In this embodiment, the axis of rotation 190 for the rotatable input is parallel to the central longitudinal axes of the first and second lead screws. The axis of rotation 190 of the rotatable input may be axially aligned with the common central longitudinal axis of the first and second elongated members, or it may be parallel to it, as depicted in FIG. 15. The teeth of internal gear 120 and spur gears 126, 180 are not shown, and instead the pitch circles of such gears are illustrated for ease.

Aspects of the present invention have been described herein with reference to certain exemplary or preferred embodiments. These embodiments are offered as merely illustrative, not limiting, of the scope of the present invention. Certain alterations or modifications which are possible include the substitution of selected features from one embodiment to another, the combination of selected features from more than one embodiment, and the elimination of certain features of described embodiments. Other alterations or modifications may be apparent to those skilled in the art in light of instant disclosure without departing from the spirit or scope of the present invention, which is defined solely with reference to the following appended claims.

1. A method of reconstraining a foreshortening self-expanding stent with a known foreshortening ratio between the crimped diameter in an intraluminal catheter based delivery system and the nominal deployed diameter in the body lumen, wherein the proximal end of the stent is in releasable fixed relation about a location along the length of an inner member of a stent delivery system, the method comprising:
translating proximally the outer member with respect to the stent at a first rate, thereby exposing at least a portion of the stent;
at the same time that the outer member is translating proximally, translating distally the inner member, thereby translating distally the proximal end of the stent, at a rate equal to the known foreshortening ratio multiplied by the first rate at which the outer member is translating proximally;
after exposing at least a length of the stent, but before translating proximally the distal end of the outer member past the proximal end of the stent, deciding to reconstrain the partially deployed stent;
subsequently translating distally the outer member with respect to the stent at a second rate, thereby reconstraining the length of the stent exposed in the previous translating proximally step; and
at the same time that the outer member is translating distally, translating proximally the inner member at a rate equal to the known foreshortening ratio multiplied by the second rate at which the outer member is translating distally.

2. A medical device delivery system comprising:
a first lead screw having a right-handed thread and a central longitudinal axis;
a second lead screw having a left-handed thread and a central longitudinal axis;
a first follower operationally coupled to the right-handed thread to translate without rotating;
a second follower operationally coupled to the left-handed thread to translate without rotating;
wherein when the first and second lead screws rotate, the first follower translates parallel to the central longitudinal axis of the first lead screw in a first linear direction and the second follower translates parallel to the central longitudinal axis of the second lead screw in a linear direction opposite the first linear direction.

3. The medical device delivery system of claim 2, wherein the central longitudinal axis of the first and second lead screws are on a common line and are coupled together to rotate about the common line in the same rotational direction and at the same time.

4. The medical device delivery system of claim 2 further comprising:
a first elongated member having a central longitudinal axis, a proximal end, a distal end, and a lumen and a total length therebetween;
a second elongated member having a central longitudinal axis, a proximal end, and a distal end, and a total length therebetween;
wherein a length of the second elongated member is disposed in the lumen of, and co-axial with, the first elongated member, and the proximal end of the first elongated member is operatively connected to the first follower, and the proximal end of the second elongated member is operatively connected to the second follower.

5. The medical device delivery system of claim 2 further comprising a self-expanding stent having a proximal end and a distal end, the stent being constrained in an initial condition to a constrained diameter in the lumen of the first elongated member near the distal end of the first elongated member, and the proximal end of the stent being fixed with respect to a position along the central longitudinal axis of the second longitudinal member

6. The medical device delivery system of claim 2, wherein the self-expanding stent foreshortens when deployed and has a nominal foreshortening ratio of the length when deployed to the nominal diameter to the length when in the constrained diameter.

7. The medical device delivery system of claim 2, wherein the ratio of the pitch of the right-handed thread to the pitch of the left-handed is equal to the nominal foreshortening ratio of the stent.

8. The medical device delivery system of claim 3 further comprising a cylinder rotatable about its central longitudinal axis operatively connected to the first and second lead screw, wherein when the cylinder is rotated about that axis, the first and second lead screws rotate about the common axis.

9. The medical device delivery system of claim 2 further comprising a housing at least partially enclosing and providing a frame for mounting the cylinder, first and second lead screws, first and second followers, and a length less than the total length of the first and second elongated members.

10. The medical device delivery system of claim 8 wherein the cylinder's central axis is axially aligned with common line of the first and second lead screws.

11. The medical device delivery system of claim 8 wherein the cylinder's central axis is parallel to the common line of the first and second lead screws.

12. The medical device delivery system of any of claim 8 wherein the cylinder's central axis is perpendicular to the common line of the first and second lead screws.

13. The medical device delivery system of claim 11 wherein the cylinder is an internal gear, and the delivery system further comprises a spur gear engaged with the internal gear, wherein the spur gear has a central axis in line with the common line of the first and second lead screws.

14. The medical device delivery system of claim 12 wherein the cylinder is a face gear, and the delivery system further comprises a spur gear engaged with the face gear, wherein the spur gear has a central axis in line with the common line of the first and second lead screws.

15. The medical device delivery system of claim 14, wherein the cylinder is a first cylinder, and the face gear includes external gear teeth around the circumference, and the delivery system further comprises an input wheel comprising a first cylinder and a second spur gear, wherein the second spur gear is engaged with the external gear teeth of the face gear, such that when the input wheel rotates the first and second lead screws rotate and the first and second followers translate.

16. The medical device delivery system of claim 9, wherein the first follower includes at least one projection external to the housing, such that the input to the system may be translation of the projection, resulting in rotation of the first and second lead screws.

17. The medical device delivery system of claim 9, wherein the first elongated member can be directly manipulated linearly in a proximal or distal direction, resulting in rotation of the first and second lead screws.

18. The medical device delivery system of claim 16, wherein the second follower is completely enclosed by the housing.

18. The medical device delivery system of claim 2, where the operative connection between the first follower and the first lead screw includes a drive bearing.

20. The medical device delivery system of claim 2, wherein the ratio of the pitch of the first lead screw to the pitch of the second lead screw is 6.5.

21. The medical device delivery system of claim 2, wherein one of the first and second lead screws is of variable pitch and the other lead screw is of constant pitch so that the drive ratio varies throughout the travel.
